(19) United States
(12) Patent Application Publication
(10) Pub. No.: US 2014/0080723 A1
(43) Pub. Date: Mar. 20, 2014

(54) Method of Fast Tuberculosis Diagnosis and Efficacy Test

(71) Applicant: Fooyin University Hospital, Pingtung County (TW)

(72) Inventors: Fu-Yen Chung, Taoyuan County (TW); Shiu-Ru Lin, Kaohsiung City (TW); Hui-Jen Chang, Pingtung County (TW); Ia-Tang Huang, Kaohsiung City (TW)

(21) Appl. No.: 13/906,924

(22) Filed: May 31, 2013

(30) Foreign Application Priority Data
Sep. 14, 2012 (TW) 101217741

(51) Int. Cl. C12Q 1/68

(52) U.S. Cl. CPC C12Q 1/6837
USPC 506/9

(57) ABSTRACT

A method is provided for fast diagnosis of tubercle bacillus (TB). The method can be used for efficacy test at the same time. 13 specific TB genes and 6 drug-resistance genes are selected. Those genes are formed into a construction for diagnosing tuberculosis and testing drug resistance simultaneously.

### TECHNICAL FIELD OF THE INVENTION

The present invention relates to tuberculosis diagnosis; more particularly, relates to using a chip array construction of specific tubercle bacillus (TB) genes and drug-resistance genes for detecting tubercle bacillus and drug resistance.

### DESCRIPTION OF THE RELATED ARTS

TB is an old contagious disease. Although it has been long on developing methods for preventing, controlling and curing this disease, TB is still a key issue in the world, which kills greatest number of people among all contagious diseases.

A characteristic of TB is that there may be no sign appeared after a person is infected and only 10% of the patients have morbidity. Most of the patients have the thalli lived in their bodies for a long time before the morbidity appears. Thus, the cause that turns a patient of latent TB into one of active TB may be exogenous reinfection or endogenous reactivation. Hence, for diagnosing TB clinically, clinical expression shown on the patients, changes shown on X-ray films and laboratorial experiments are all required for confirmation.

Regarding laboratorial examination, technologies relating to histopathology, staining of acid-fast bacterium and TB culturing are used. However, they all have their limits. Take staining of acid-fast bacterium as an example. At least 5000 to 10000 bacteria have to be contained in one milli-liter of a specimen. Besides, there exists a high possibility of fake positive for this method. That is because some other bacteria may show positive results too. Regarding TB culturing, although it is the most sensitive diagnosing method, it takes 4 to 8 weeks to obtain the result and is not suitable for clinical use.

As following development of biological technologies, clinical diagnosis of TB has evolutional progress on molecular diagnostic technologies, like polymerase chain reaction (PCR), PCR-Restriction Fragment Length Polymorphism (PCR-RFLP), etc. In the early years, PCR are directly used for detecting molecular marks of TB in patients' specimens, like deoxyribonucleic acid (DNA) or ribonucleic acid (RNA) of heat shock proteins 65 (hsp65) and inserted section 6110 (IS6110). In addition, with coordination of restriction fragment length polymorphism (RFLP), the nucleic-acid molecular typing of the detected thallus is identified. However, expression of specific DNA or messenger ribonucleic acid (mRNA) in a patient's specimen is still not satisfactory clinically.

Through decoding the TB genes, it is found that genomic deletion is existed between TB and mycobacterium bovis BCG, where lost sections are called regions-of-difference (RD). The causes for these RDs may be errors on duplicating DNAs of the genes or on inserting sections, including deletion, insertion, inversion, replication, etc.

The RD sections have many important genes and pathogenic factors. These sections may differ between pathogens of TB genes. Hence, the RD sections can be used for identifying the TB genes, which identifies specific genes with high sensitivity.

In 2009, 14 specific target genes were selected as testing targets for constructing a TB gene diagnosis chip. Through using a platform for detecting tiny amount of nucleic acid, multiple gene targets are detected simultaneously, where sensitivity reaches a level for detection with only 5 cells in one milli-liter of blood. Thus, a TB gene detection chip for sputum specimen is constructed.

This chip can detect 85% of TB complex (TBC), where PCR-RFLP is 62.5%. In an experiment, 52 specimens are picked out by the chip from 56 positive-cultured and positive-dyed sputum specimens, where only 39 are picked out through PCR-RFLP. In another experiment, 16 specimens are picked out by the chip from 24 positive-cultured and negative-dyed sputum specimens, where only 11 are picked out through PCR-RFLP.

Accordingly, gene chip detection is easily operated without much human labor and time. Moreover, sensitivity of the gene chip detection is far higher than PCR-RFLP.

In the market, some TB detection sets include Spoligotyping Method (Holland), TB Ag Rapid Test (Taiwan), Amplified MTDR (USA), DR. MTBC Screen Kit (Taiwan) and GenoType MTBDRplus (German). Therein, Spoligotyping Method detects TB oligonucleotide spectrum at first and, then, finds its typing from a database. But, the resolving power is low and drug resistance is not detected. TB Ag Rapid Test uses specific antigen to detect TB directly. But, its cost is high; TB colony has to be cultured; its procedure is complex; it takes time; and, not to mention, drug resistance is not detected. DR. MTBC Screen Kit magnifies specific gene sections through PCR and, then, the chip is processed through hybridization. Although drug-resistance genes against Rifampicin can be found, 100-thousand TB bacteria in one milli-liter of sputum are required for valid detection with a 65% sensitivity only. GenoType MTBDRplus magnifies TB drug-resistance genes through PCR and, then, hybridization is processed with probes. Although drug-resistance genes in TB against Ofloxacin, Streptomycin and Ethambutol can be found, its cost is high; its technology is complex; it takes time for detection; and it only tests efficacy but not TB itself.

Furthermore, a prior art of detection chip directly detected active TB in a sputum specimen. Yet, it detects Tb only and do not analyzes efficacy on gene cluster. Hence, the prior arts do not fulfill all users' requests on actual use.

### SUMMARY OF THE INVENTION

The main purpose of the present invention is to use a construction of specific TB genes and drug-resistance genes for detecting TB and testing drug resistance simultaneously.

To achieve the above purpose, the present invention is a method of fast tuberculosis diagnosis and efficacy test, comprising steps of: (a) obtaining a sputum specimen and extracting messenger ribonucleic acids (mRNAs) in the sputum specimen to synthesize a required amount of complementary deoxyribonucleic acids (cDNAs) through reverse transcription; (b) labeling the cDNAs with Biotin to obtain a plurality of bioprobes; (c) synthesizing TB genes and drug-resistance genes in vitro into a specific gene cluster of TB and a drug-resistance gene cluster and obtaining a chip array construction through crosslinking by dotting the specific gene cluster of TB, the drug-resistance gene cluster, positive controls, negative controls and blank controls into array on a nylon membrane, where the specific gene cluster of TB is specified through a specific oligonucleotide design; and where the chip array construction is formed into a plurality of gene-testing points on the nylon membrane; (d) hybridizing the gene-testing points of the chip array construction with biomolecules of the bioprobes and washing out un-hybridized bioprobes; and (e) blocking the bioprobes obtained after hybridization of the chip array construction to form crosslinks with Streptavidin-HRP accompanied with a washing process afterwards and, then, adding a coloring agent of diaminobenzidine (DAB) to process color development for analyzing and interpreting an image thus obtained. Accordingly, a novel method of fast tuberculosis diagnosis and efficacy test is obtained.

### BRIEF DESCRIPTIONS OF THE DRAWINGS

The present invention will be better understood from the following detailed description of the preferred embodiment according to the present invention, taken in conjunction with the accompanying drawings, in which

FIG. 1 is the flow view showing the preferred embodiment according to the present invention;

FIG. 2 is the view showing the testing areas;

FIG. 3 is the view showing the gene arrangements;

FIG. 4 is the view showing the interpretation of the bacillus tuberculosis testing; and

FIG. 5 is the view showing the interpretation of the drug resistance testing.

### DESCRIPTION OF THE PREFERRED EMBODIMENT

The following description of the preferred embodiment is provided to understand the features and the structures of the present invention.

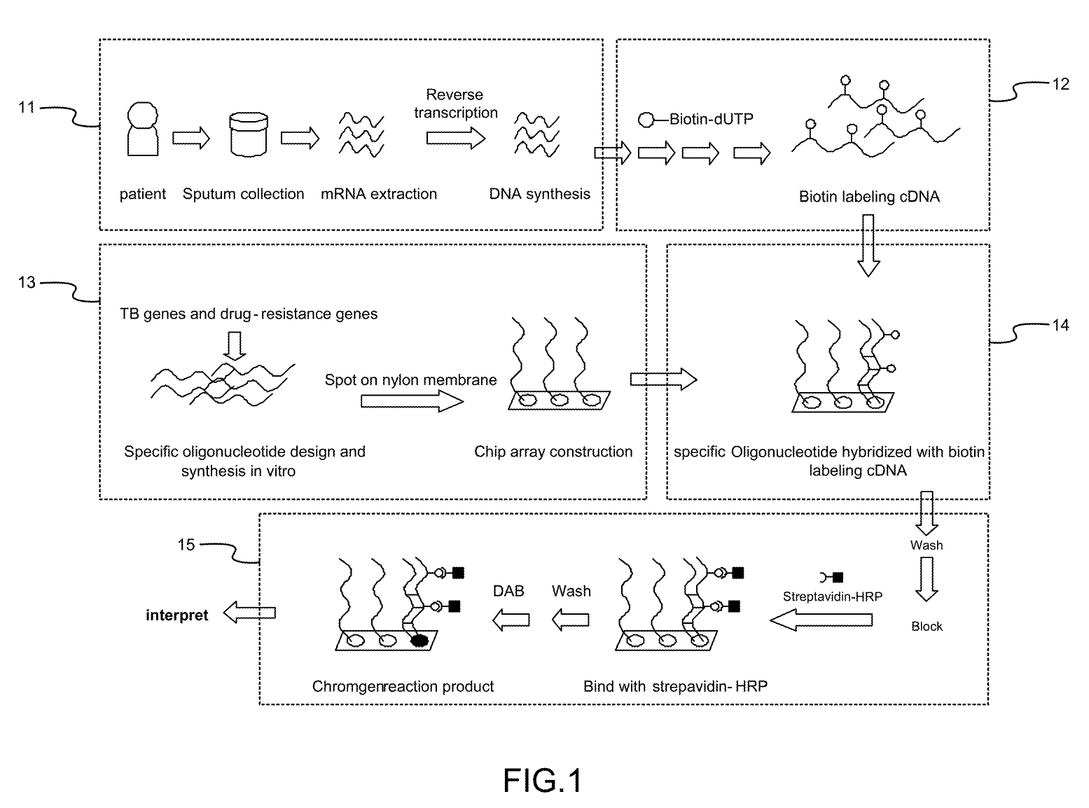

Please refer to FIG. 1, which is a flow view showing a preferred embodiment according to the present invention. As shown in the figure, the present invention is a method of fast tuberculosis diagnosis and efficacy test, comprising the following steps:

(a) DNA extraction 11: A sputum specimen of a patient is collected and messenger ribonucleic acids (mRNAs) in the sputum specimen is extracted for synthesizing a required amount of complementary deoxyribonucleic acids (cDNAs) through reverse transcription.

(b) Multiple linear amplification and labeling 12: The cDNAs are labeled with Biotin to form a plurality of bioprobes.

(c) Fabrication of chip array construction 13: Tubercle bacillus (TB) genes along with drug-resistance genes are synthesized in vitro into a specific gene cluster of TB along with a drug-resistance gene cluster, where the specific gene cluster of TB is specified through a specific oligonucleotide design. Then, a chip array construction is formed through crosslinking by dotting the specific gene cluster of TB, the drug-resistance gene cluster, positive controls, negative controls and blank controls into array on a nylon membrane. Therein, the specific gene cluster of TB comprises 13 specific TB genes; the drug-resistance gene cluster comprises 6 drug-resistance genes; and the chip array construction is formed into a plurality of gene-testing points on the nylon membrane.

(d) Hybridization 14: The bioprobes are hybridized with the chip array construction, where the gene-testing points of the chip array construction are hybridized with biomolecules of the labeled bioprobes. Then, un-hybridized bioprobes are washed out.

(e) Color development 15: After hybridization with the chip array construction, the bioprobes are blocked to form crosslinks with Streptavidin-HRP accompanied with a washing process afterwards. Then, a coloring agent of diaminobenzidine (DAB) is added for color development to analyze and interpret an image thus obtained.

As shown in Table 1, the specific gene cluster of TB comprises specific oligonucleotide sequences selected from the specific TB genes. These 13 specific TB genes comprises hsp65, Rv0577, Rv3120, Rv2073c, Rv1970, Rv3875, Rv3347c, Rv1510, Rv0186, Rv0124, TbD1, mtp40 and mpb83, which are obtained through analysis by Primer Premier 5.0 (PREMIER Biosoft International, Palo Alto, Calif.).

TABLE 1

| | Gene | |
|---|---|---|
| No. | Name | Oligonucleotide Sequence |
| 1 | hsp65 | CAT CGG TCT TCT TGG CTA CCT CTT TGA CCA GCT CG |
| 2 | Rv0577 | CGT CGT AAC CCC AGC CGA ACA ACG ATG TGT AGA AC |
| 3 | Rv3120 | CGG ATG CCA GAA TAG TCG GCA AAG TAC CAG AGC A |
| 4 | Rv2073c | GCC GGC TTT GGC CGA TCC GTA GAC ATA GTT G |
| 5 | Rv1970 | GTC ACC GGA CTG GTT GTT GAG GTA TGC GGT G |
| 6 | Rv3875 | CTT CCC CTC GTC AAG GAG GGA ATG AAT GGA CGT G |
| 7 | Rv3347c | GTG TTG TAG CTG CCC GAG TTG AAT ACC CCG AAG TT |
| 8 | Rv1510 | CCA GAT AGA TGA CCG TGT AGA CGC AGG CAA CGG |
| 9 | Rv0186 | GGT CCT CGG AAA GGT ACT CGA AGT TGC GGC |
| 10 | Rv0124 | CGT CTG CAC GAA CTG CTG ATG AAA CGC CG |
| 11 | TbD1 | TCG GCT GCT CGG TCC CTC TGA TAC TTG AGA TTC TG |
| 12 | mtp40 | ATC CGC AGT GAT GCC AAC TCA GGA AAC CAC AC |
| 13 | mpb83 | GAG GTC AGG GTA CTG AGC ATC GGG TTG TTG GAA G |

As shown in Table 2, the drug-resistance gene cluster for testing anti-tuberculosis drugs comprises 6 oligonucleotide sequences, which comprises katG, rpoB, gyrA, embB, rpsL and rrs.

TABLE 2

| | Oligonucleotide | |
|---|---|---|
| Drug | Name | Oligonucleotide sequence (5′ to 3′) |
| Isoniazid | katG-W1 | AAC TAG CTG TGA GAC AGT CAA TCC CGA TGC CCG |
| | katG-W315 | CGA TGC CGC TGG TGA TCG CGT CCT TA |
| | katG-Q315 | CGA TGC CGC TGG TGA TCG TGT CCT TA |
| Rifampicin | rpoB-W1 | GAC TCG GAC TAG GAC TAG CGG CTG TTT TGC TCT |
| | rpoB-W450 | CCC TCA GGG GTT TCG ATC GGG CAC AT |
| | rpoB-Q450 | CCC TCA GGG GTT TCG ATC GAG CAC AT |
| | rpoB-W513 | TCG ACC ACC TTG CGG TAC GGC GTT TC |
| | rpoB-Q513 | TCG ACC ACC TTG CGG TAC GGA GTT TC |
| | rpoB-W522 | GTA CAC GAT CTC GTC GCT AAC CAC GCC GT |
| | rpoB-Q522 | GTA CAC GAT CTC GTC GCT AAC TAC GCC GT |
| | rpoB-W526 | GTC GGC GGT CAG GTA CAC GAT CTC GT |
| | rpoB-Q526 | GTC GGC GGT CAG GTA CAT GAT CTC GT |
| | rpoB-W529 | TCC TCC TCG TCG GCG CTC AGG TAC A |
| | rpoB-Q529 | TCC TCC TCG TCG GAG CTC AGG TAC A |
| | rpoB-W531 | CCA CCA CGT GGC GGT CCT C |
| | rpoB-Q531 | CCA CTA CGT GGC GGT CCT C |
| Ofloxacin | gyrA-W1 | CGG GAA TCC TCT TCT ACC TCA ACA ACT CCG CGC |
| | gyrA-W80 | CCC ATG GTC TCG GCA ACC GAC CG |
| | gyrA-Q80 | CCC ATG GTC TCG GCA ACT GAC CG |
| | gyrA-W88-91 | CGT AGA TCG ACG CGT CGC CGT GC |
| | gyrA-Q88-91 | CGT ATA TCG ACG CGT CGC CGT GC |
| | gyrA-W94 | GCC ATG CGC ACC AGG CTG TCG TAG AT |
| | gyrA-Q94 | GCC ATG CTC ACC AGG CTG TCG TAG AT |
| Ethambutol | embB-W1 | GTG TCC AGC TTC TTA GCC GAG TAG TCC GGT GT |
| | embB-W306 | CGG GCC ATG CCC AGG ATG TAG CC |
| | embB-Q306 | CGG GCC ATG CCC AGG ATA TAG CC |
| | embB-W319 | GGG CTG CCG AAC CAG CGG AAA TAG TTG G |
| | embB-Q319 | GGG CTG TCG AAC CAG CGG AAA TAG TTG G |
| | embB-W406 | CGA GCG CGA TGA TGC CCT CCG |
| | embB-Q406 | CGA GCT CGA TGA TGC CCT CCG |
| Streptomycin | rpsL-W1 | GCG GTC TTG ACC TTA CTG ATC TTG TCC CGA |
| | rpsL-W43 | GAA GCG CCG AGT TCG GCT TCT TCG GAG |
| | rpsL-Q43 | GAA GCG TCG AGT TCG GCT TCT TCG GAG |
| | rpsL-W88 | GCA CAC CAG GCA GGT CCT TCA CCC |
| | rpsL-Q88 | GCA CAC TAG GCA GGT CCT TCA CCC |
| Streptomycin | rrs-W1 | CGT AGG AGT CTG GGC CGT ATC TCA GTC CCA |
| | rrs-W513 | CCT ACG TAT TAC CGC GGC TGC TGG CA |
| | rrs-Q513 | CCT ACT TAT TAC CGC GGC TGC TGG CA |
| | rrs-W514 | GCA CCC TAC GTA TTA CCG CGG CTG CT |
| | rrs-Q514 | GCA CTC TAC GTA TTA CCG CGG CTG CT |
| | rrs-W1401 | TGA CGT GAC GGG CGG TGT GTA CAA GG |
| | rrs-Q1401 | TGA CGT GAC GGG CGG TAT GTA CAA GG |
| | rrs-W1484 | GAC TTC GTC CCA ATC GCC GAT CCC ACC TTC |
| | rrs-Q1484 | GAC TTC GTC CCA ATC GCC GAT CCT ACC TTC |
| Positive control | rrl | GTG TTA CCA CTG ACT GGT ACG GCT ACC TTC CTG |

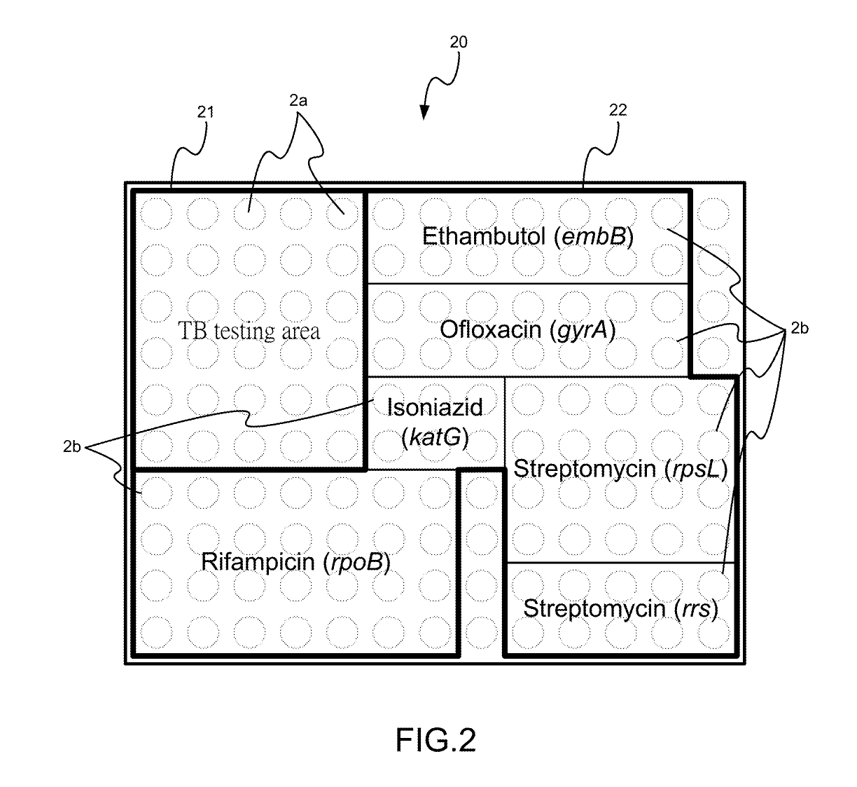

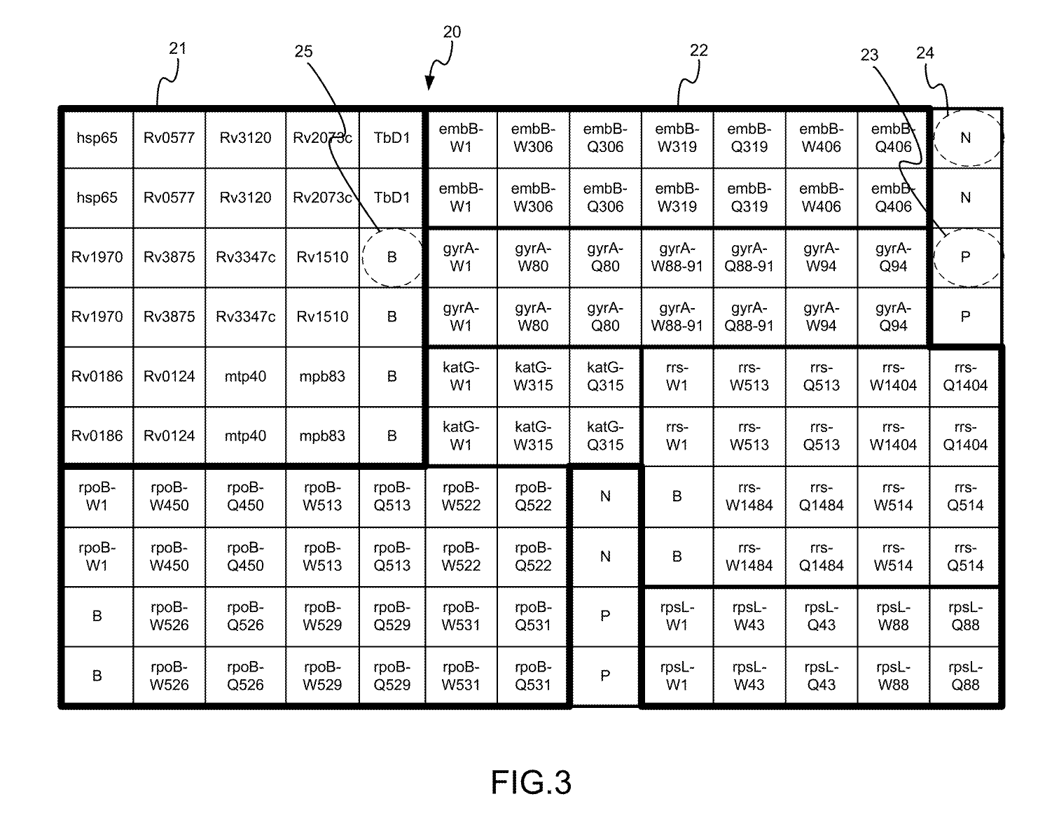

Please refer to FIG. 2 and FIG. 3, which are views showing testing areas and gene arrangements. As shown in the figures, a chip array construction 20 comprises a testing area of bacillus tuberculosis 21 and a testing area of drug resistance 22. In FIG. 2, P is a positive control 23, N is a negative control 24 and B is a blank control 25.

The testing area of bacillus tuberculosis 21 comprises a plurality of gene-testing points 2a for separately conjugating a specific gene cluster of TB with specific bioprobes to be reacted with specific biomolecules of the specific bioprobes for color development. This specific gene cluster of TB comprises 13 specific TB genes, which are hsp65, Rv0577, Rv3120, Rv2073c, Rv1970, Rv3875, Rv3347c, Rv1510, Rv0186, Rv0124, TbD1, mtp40 and mpb83.

The testing area of drug resistance 22 has a plurality of gene-testing points conjugated with a drug-resistance gene cluster to be reacted with anti-tuberculosis drugs of Isoniazid, Rifampicin, Ofloxacin, Ethambutol and Streptomycin for color development. The conjugated drug-resistance gene cluster comprises 6 drug-resistance genes, which are katG, rpoB, gyrA, embB, rpsL and rrs.

The above gene-testing points 2a.2b are arranged into array.

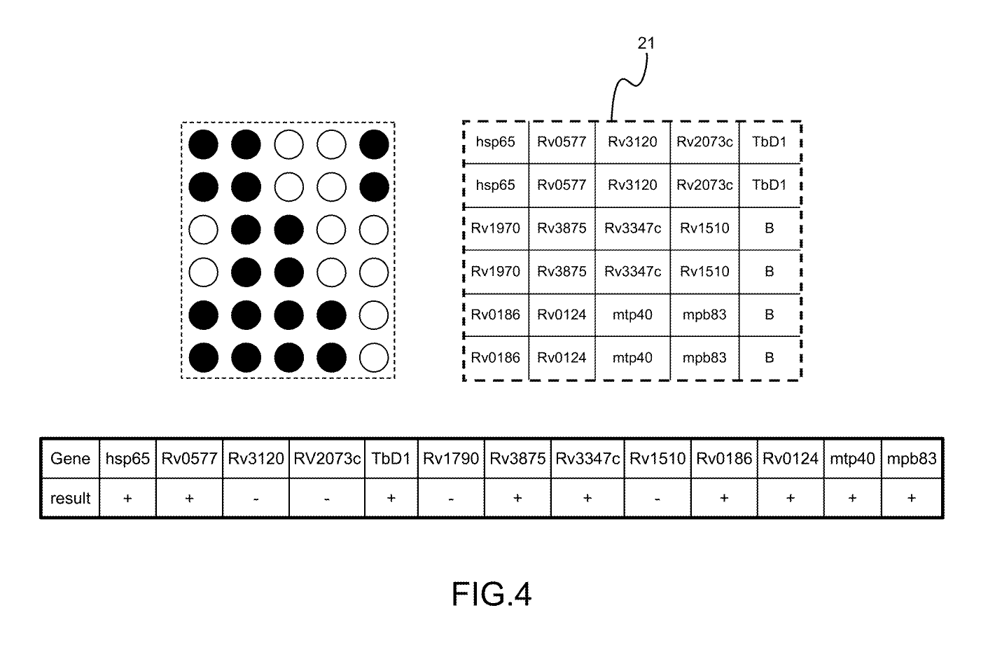

Please refer to FIG. 4, which is a view showing an interpretation of the bacillus tuberculosis testing. As shown in the figure, 13 specific TB genes and 6 drug-resistance genes are arranged in array on a nylon membrane to form a chip array construction. Therein, a testing area of bacillus tuberculosis 21 is processed through color development. If a color is developed, a specific gene is detected by expression for identification. In the figure, a result of color development for the chip array construction are as follows: hsp65(+), Rv0577(+), Rv31 20(−), Rv2073c(−), TbD1 (+), Rv1970(−), Rv3875(+), Rv3347c(+), Rv1510(−), Rv0186(+), Rv0124(+), mtp40(+) and mpb83(+). For interpretation, the sign (+) means positive reaction. More detailed comparison is shown in the following Table 3.

TABLE 3

| | hsp65 | Rv0577 | Rv3120 | Rv2073c | TbD1 | Rv1970 | Rv3875 | Rv3347c | Rv1510 | Rv0186 | Rv0124 | mtp40 | mpb83 |
|---|---|---|---|---|---|---|---|---|---|---|---|---|---|
| Organisms other than | − | − | | | | | | | | | | | |
| Mycobacterium | | | | | | | | | | | | | |
| NTM* | + | − | | | | | | | | | | | |
| M. canettii | + | + | − | + | + | + | + | + | + | + | + | + | + |
| M. tuberculosis | + | + | + | + | − | + | + | + | + | + | + | + | + |
| M. africamun(lb) | + | + | + | − | + | + | + | + | + | + | + | + | + |
| Oryx bacillus | + | + | + | − | + | − | + | + | + | + | + | + | + |
| M. africamun(lib) | + | + | + | − | +/− | − | + | + | + | − | − | + | + |
| Dassiebacillun | + | + | + | − | + | − | − | + | + | + | + | + | + |
| M. microti | + | + | + | − | + | − | − | − | + | + | + | + | + |
| M. caprie | + | + | − | − | + | − | + | + | + | + | + | + | + |
| M. bovis | + | + | − | − | + | − | + | + | − | + | + | + | + |
| M. bovis BCG | + | + | − | − | + | − | − | + | − | + | + | + | + |

*NTM: Nontuberculous Mycobacterium

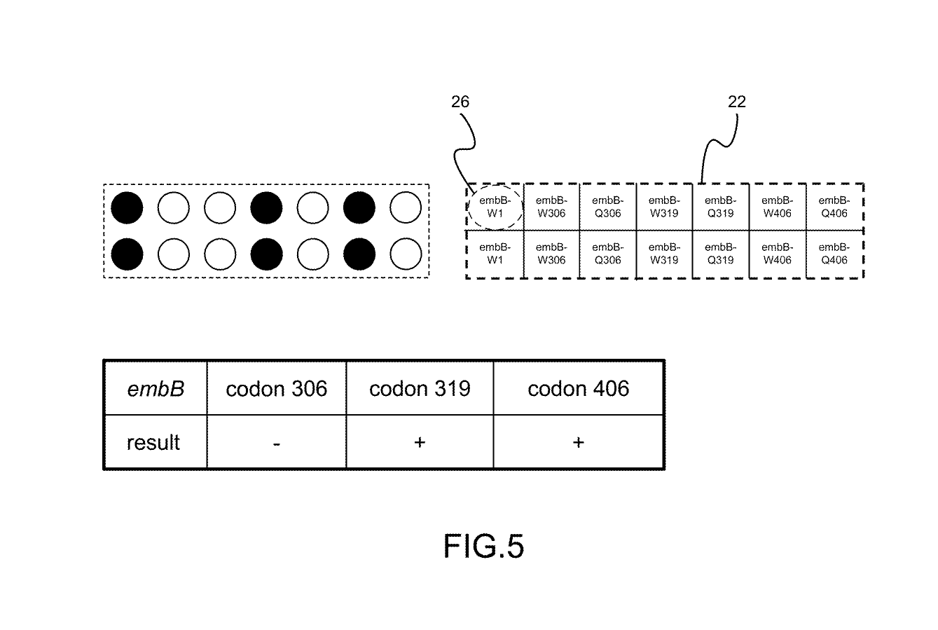

Please refer to FIG. 5, which is a view showing an interpretation of the drug resistance testing. As shown in the figure, 13 specific TB genes and 6 drug-resistance genes are arranged to form a chip array construction on a nylon membrane. Therein, a testing area of drug resistance 22 is used for testing Ethambutol. EmbB-W1 is set as a positive control to develop color for embB; embB-W306, embB-W319 and embB-W406 are wild-type probes for embB codon 306, 319 and 406; and, embB-Q306, embB-Q319 and embB-Q406 are inner controls for embB codon 306, 319 and 406 in easily-mutating positions., when the gene is mutated and is not connected to the wild-type probe, the color is not developed and, thus, mutation of the drug-resistance gene is analyzed. A result is shown as follows: codon 306 (+), codon 319 (+) and codon 306 (+). Interpretation made for the result is that embB codon 306 is mutated, which shows this gene has drug resistance to Ethambutol.

To sum up, the present invention is a method of fast tuberculosis diagnosis and efficacy test, where specific TB genes and drug-resistance genes are used as probes to test TB and drug resistance simultaneously through analysis after hybridization; and, thus, the present invention is a fast method with low cost for detecting TB and testing drug resistance simultaneously.

1. A method of fast tuberculosis diagnosis and efficacy test, comprising steps of:
(a) obtaining a sputum specimen and extracting messenger ribonucleic acids (mRNAs) in said sputum specimen to synthesize a required amount of complementary deoxyribonucleic acids (cDNAs) through reverse transcription;
(b) labeling said cDNAs with Biotin to obtain a plurality of bioprobes;
(c) synthesizing tubercle bacillus (TB) genes and drug-resistance genes in vitro into a specific gene cluster of TB and a drug-resistance gene cluster and obtaining a chip array construction through crosslinking by dotting said specific gene cluster of TB, said drug-resistance gene cluster, positive controls, negative controls and blank controls into array on a nylon membrane,
wherein said specific gene cluster of TB is specified through a specific oligonucleotide design; and
wherein said chip array construction is formed into a plurality of gene-testing points on said nylon membrane;
(d) hybridizing said gene-testing points of said chip array construction with biomolecules of said bioprobes and washing out un-hybridized bioprobes; and
(e) blocking said bioprobes obtained after hybridization of said chip array construction to form crosslinks with Streptavidin-HRP accompanied with a washing process afterwards and, then, adding a coloring agent to process color development to analyze and interpret an image thus obtained.

2. The method according to claim 1, wherein said specific gene cluster of TB comprises 13 specific TB genes; wherein said 13 specific TB genes comprises hsp65, Rv0577, Rv3120, Rv2073c, Rv1970, Rv3875, Rv3347c, Rv1510, Rv0186, Rv0124, TbD1, mtp40 and mpb83; and
wherein said specific TB genes are reacted with biomolecules of said specific bioprobes to develop colors.

3. The method according to claim 2, wherein hsp65 of said specific gene cluster of TB has a oligonucleotide array sequence and said oligonucleotide array sequence has gene identifiers as follows: CAT CGG TCT TCT TGG CTA CCT CTT TGA CCA GCT CG (SEQ ID NO:1).

4. The method according to claim 2, wherein Rv0577 of said specific gene cluster of TB has a oligonucleotide array sequence and said oligonucleotide array sequence has gene identifiers as follows: CGT CGT AAC CCC AGC CGA ACA ACG ATG TGT AGA AC (SEQ ID NO:2).

5. The method according to claim 2, wherein Rv3120 of said specific gene cluster of TB has a oligonucleotide array sequence and said oligonucleotide array sequence has gene identifiers as follows: CGG ATG CCA GAA TAG TCG GCA AAG TAC CAG AGC A (SEQ ID NO:3).

6. The method according to claim 2, wherein Rv2073c of said specific gene cluster of TB has a oligonucleotide array sequence and said oligonucleotide array sequence has gene identifiers as follows: GCC GGC TTT GGC CGA TCC GTA GAC ATA GTT G (SEQ ID NO:4).

7. The method according to claim 2, wherein Rv1970 of said specific gene cluster of TB has a oligonucleotide array sequence and said oligonucleotide array sequence has gene identifiers as follows: GTC ACC GGA CTG GTT GTT GAG GTA TGC GGT G (SEQ ID NO:5).

8. The method according to claim 2, wherein Rv3875 of said specific gene cluster of TB has a oligonucleotide array sequence and said oligonucleotide array sequence has gene identifiers as follows: CTT CCC CTC GTC AAG GAG GGA ATG AAT GGA CGT G (SEQ ID NO:6).

9. The method according to claim 2, wherein Rv3347c of said specific gene cluster of TB has a oligonucleotide array sequence and said oligonucleotide array sequence has gene identifiers as follows: GTG TTG TAG CTG CCC GAG TTG AAT ACC CCG AAG TT (SEQ ID NO:7).

10. The method according to claim 2, wherein Rv1510 of said specific gene cluster of TB has a oligonucleotide array sequence and said oligonucleotide array sequence has gene identifiers as follows: CCA GAT AGA TGA CCG TGT AGA CGC AGG CAA CGG (SEQ ID NO:8).

11. The method according to claim 2, wherein Rv0186 of said specific gene cluster of TB has a oligonucleotide array sequence and said oligonucleotide array sequence has gene identifiers as follows: GGT CCT CGG AAA GGT ACT CGA AGT TGC GGC (SEQ ID NO:9).

12. The method according to claim 2, wherein Rv0124 of said specific gene cluster of TB has a oligonucleotide array sequence and said oligonucleotide array sequence has gene identifiers as follows: CGT CTG CAC GAA CTG CTG ATG AAA CGC CG (SEQ ID NO:10).

13. The method according to claim 2, wherein TbD1 of said specific gene cluster of TB has a oligonucleotide array sequence and said oligonucleotide array sequence has gene identifiers as follows: TCG GCT GCT CGG TCC CTC TGA TAC TTG AGA TTC TG (SEQ ID NO:11).

14. The method according to claim 2, wherein mtp40 of said specific gene cluster of TB has a oligonucleotide array sequence and said oligonucleotide array sequence has gene identifiers as follows: ATC CGC AGT GAT GCC AAC TCA GGA AAC CAC AC (SEQ ID NO:12).

15. The method according to claim 2, wherein mpb83 of said specific gene cluster of TB has a oligonucleotide array sequence and said oligonucleotide array sequence has gene identifiers as follows: GAG GTC AGG GTA CTG AGC ATC GGG TTG TTG GAA G (SEQ ID NO:13).

16. The method according to claim 1, wherein said drug-resistance gene cluster comprises 6 drug-resistance genes;
wherein said 6 drug-resistance genes comprises katG, rpoB, gyrA, embB, rpsL and rrs; wherein said drug-resistance genes are reacted with anti-tuberculosis drugs to develop colors; and wherein said anti-tuberculosis drugs comprises Isoniazid, Rifampicin, Ofloxacin, Ethambutol and Streptomycin.

17. The method according to claim 1, wherein said gene-testing points are arranged into array.

18. The method according to claim 1, wherein said chip array construction comprises
a first testing area, said first testing area being an area to test bacillus tuberculosis, said first testing area comprising a plurality of first gene-testing points, said first gene-testing points comprising said specific gene cluster of TB conjugated with said bioprobes to be reacted with biomolecules of said bioprobes to process color development, said specific gene cluster of TB comprising 13 specific TB genes, said 13 specific TB genes comprising hsp65, Rv0577, Rv3120, Rv2073c, Rv1970, Rv3875, Rv3347c, Rv1510, Rv0186, Rv0124, TbD1, mtp40 and mpb83; and
a second testing area, said second testing area being an area to test drug resistance, said second testing area comprising a plurality of second gene-testing points, said second gene-testing points comprising said drug-resistance gene cluster to be reacted with anti-tuberculosis drugs of Isoniazid, Rifampicin, Ofloxacin, Ethambutol and Streptomycin to process color development, said drug-resistance gene cluster comprising 6 drug-resistance genes, said 6 drug-resistance genes comprising katG, rpoB, gyrA, embB, rpsL and rrs.

19. The method according to claim 18, wherein said chip array construction further comprises positive controls, negative controls and blank controls.


SEQUENCE LISTING (58 sequences)

SEQ ID NO: 1 - DNA, 35 bp, Artificial Sequence
  hsp65 Oligonucleotide
catcggtcttcttggctacctctttgaccagctcg

SEQ ID NO: 2 - DNA, 35 bp, Artificial Sequence
  Rv0577 Oligonucleotide
cgtcgtaaccccagccgaacaacgatgtgtagaac

SEQ ID NO: 3 - DNA, 34 bp, Artificial Sequence
  Rv3120 Oligonucleotide
cggatgccagaatagtcggcaaagtaccagagca

SEQ ID NO: 4 - DNA, 31 bp, Artificial Sequence
  Rv2073c Oligonucleotide
gccggctttggccgatccgtagacatagttg

SEQ ID NO: 5 - DNA, 31 bp, Artificial Sequence
  Rv1970 Oligonucleotide
gtcaccggactggttgttgaggtatgcggtg

SEQ ID NO: 6 - DNA, 34 bp, Artificial Sequence
  Rv3875 Oligonucleotide
cttcccctcgtcaaggagggaatgaatggacgtg

SEQ ID NO: 7 - DNA, 35 bp, Artificial Sequence
  Rv3347c Oligonucleotide
gtgttgtagctgcccgagttgaataccccgaagtt

SEQ ID NO: 8 - DNA, 33 bp, Artificial Sequence
  Rv1510 Oligonucleotide
ccagatagatgaccgtgtagacgcaggcaacgg

SEQ ID NO: 9 - DNA, 30 bp, Artificial Sequence
  Rv0186 Oligonucleotide
ggtcctcggaaaggtactcgaagttgcggc

SEQ ID NO: 10 - DNA, 29 bp, Artificial Sequence
  Rv0124 Oligonucleotide
cgtctgcacgaactgctgatgaaacgccg

SEQ ID NO: 11 - DNA, 35 bp, Artificial Sequence
  TbD1 Oligonucleotide
tcggctgctcggtccctctgatacttgagattctg

SEQ ID NO: 12 - DNA, 32 bp, Artificial Sequence
  mtp40 Oligonucleotide
atccgcagtgatgccaactcaggaaaccacac

SEQ ID NO: 13 - DNA, 34 bp, Artificial Sequence
  mpb83 Oligonucleotide
gaggtcagggtactgagcatcgggttgttggaag

SEQ ID NO: 14 - DNA, 33 bp, Artificial Sequence
  katG-W1 Oligonucleotide
aactagctgtgagacagtcaatcccgatgcccg

SEQ ID NO: 15 - DNA, 26 bp, Artificial Sequence
  katG-W315 Oligonucleotide
cgatgccgctggtgatcgcgtcctta

SEQ ID NO: 16 - DNA, 26 bp, Artificial Sequence
  katG-Q315 Oligonucleotide
cgatgccgctggtgatcgtgtcctta

SEQ ID NO: 17 - DNA, 33 bp, Artificial Sequence
  rpoB-W1 Oligonucleotide
gactcggactaggactagcggctgttttgctct

SEQ ID NO: 18 - DNA, 26 bp, Artificial Sequence
  rpoB-W450 Oligonucleotide
ccctcaggggtttcgatcgggcacat

SEQ ID NO: 19 - DNA, 26 bp, Artificial Sequence
  rpoB-Q450 Oligonucleotide
ccctcaggggtttcgatcgagcacat

SEQ ID NO: 20 - DNA, 26 bp, Artificial Sequence
  rpoB-W513 Oligonucleotide
tcgaccaccttgcggtacggcgtttc

SEQ ID NO: 21 - DNA, 26 bp, Artificial Sequence
  rpoB-Q513 Oligonucleotide
tcgaccaccttgcggtacggagtttc

SEQ ID NO: 22 - DNA, 29 bp, Artificial Sequence
  rpoB-W522 Oligonucleotide
gtacacgatctcgtcgctaaccacgccgt

SEQ ID NO: 23 - DNA, 29 bp, Artificial Sequence
  rpoB-Q522 Oligonucleotide
gtacacgatctcgtcgctaactacgccgt

SEQ ID NO: 24 - DNA, 26 bp, Artificial Sequence
  rpoB-W526 Oligonucleotide
gtcggcggtcaggtacacgatctcgt

SEQ ID NO: 25 - DNA, 26 bp, Artificial Sequence
  rpoB-Q526 Oligonucleotide
gtcggcggtcaggtacatgatctcgt

SEQ ID NO: 26 - DNA, 25 bp, Artificial Sequence
  rpoB-W529 Oligonucleotide
tcctcctcgtcggcggtcaggtaca

SEQ ID NO: 27 - DNA, 25 bp, Artificial Sequence
  rpoB-Q529 Oligonucleotide
tcctcctcgtcggaggtcaggtaca

SEQ ID NO: 28 - DNA, 22 bp, Artificial Sequence
  rpoB-W531 Oligonucleotide
ccaccacgtggcggtcctcctc

SEQ ID NO: 29 - DNA, 22 bp, Artificial Sequence
  rpoB-Q531 Oligonucleotide
ccactacgtggcggtcctcctc

SEQ ID NO: 30 - DNA, 33 bp, Artificial Sequence
  gyrA-W1 Oligonucleotide
cgggaatcctcttctacctcaacaactccgcgc

SEQ ID NO: 31 - DNA, 23 bp, Artificial Sequence
  gyrA-W80 Oligonucleotide
cccatggtctcggcaaccgaccg

SEQ ID NO: 32 - DNA, 23 bp, Artificial Sequence
  gyrA-Q80 Oligonucleotide
cccatggtctcggcaactgaccg

SEQ ID NO: 33 - DNA, 23 bp, Artificial Sequence
  gyrA-W88-91 Oligonucleotide
cgtagatcgacgcgtcgccgtgc

SEQ ID NO: 34 - DNA, 23 bp, Artificial Sequence
  gyrA-Q88-91 Oligonucleotide
cgtatatcgacgcgtcgccgtgc

SEQ ID NO: 35 - DNA, 26 bp, Artificial Sequence
  gyrA-W94 Oligonucleotide
gccatgcgcaccaggctgtcgtagat

SEQ ID NO: 36 - DNA, 26 bp, Artificial Sequence
  gyrA-Q94 Oligonucleotide
gccatgctcaccaggctgtcgtagat

SEQ ID NO: 37 - DNA, 32 bp, Artificial Sequence
  embB-W1 Oligonucleotide
gtgtccagcttcttagccgagtagtccggtgt

SEQ ID NO: 38 - DNA, 23 bp, Artificial Sequence
  embB-W306 Oligonucleotide
cgggccatgcccaggatgtagcc

SEQ ID NO: 39 - DNA, 23 bp, Artificial Sequence
  embB-Q306 Oligonucleotide
cgggccatgcccaggatatagcc

SEQ ID NO: 40 - DNA, 28 bp, Artificial Sequence
  embB-W319 Oligonucleotide
gggctgccgaaccagcggaaatagttgg

SEQ ID NO: 41 - DNA, 28 bp, Artificial Sequence
  embB-Q319 Oligonucleotide
gggctgtcgaaccagcggaaatagttgg

SEQ ID NO: 42 - DNA, 21 bp, Artificial Sequence
  embB-W406 Oligonucleotide
cgagcgcgatgatgccctccg

SEQ ID NO: 43 - DNA, 21 bp, Artificial Sequence
  embB-Q406 Oligonucleotide
cgagctcgatgatgccctccg

SEQ ID NO: 44 - DNA, 33 bp, Artificial Sequence
  rpsL-W1 Oligonucleotide
gcggtcttgaccttactgatcttgtcccgacga

SEQ ID NO: 45 - DNA, 27 bp, Artificial Sequence
  rpsL-W43 Oligonucleotide
gaagcgccgagttcggcttcttcggag

SEQ ID NO: 46 - DNA, 27 bp, Artificial Sequence
  rpsL-Q43 Oligonucleotide
gaagcgtcgagttcggcttcttcggag

SEQ ID NO: 47 - DNA, 24 bp, Artificial Sequence
  rpsL-W88 Oligonucleotide
gcacaccaggcaggtccttcaccc

SEQ ID NO: 48 - DNA, 24 bp, Artificial Sequence
  rpsL-Q88 Oligonucleotide
gcacactaggcaggtccttcaccc

SEQ ID NO: 49 - DNA, 30 bp, Artificial Sequence
  rrs-W1 Oligonucleotide
cgtaggagtctgggccgtatctcagtccca

SEQ ID NO: 50 - DNA, 26 bp, Artificial Sequence
  rrs-W513 Oligonucleotide
cctacgtattaccgcggctgctggca

SEQ ID NO: 51 - DNA, 26 bp, Artificial Sequence
  rrs-Q513 Oligonucleotide
cctacttattaccgcggctgctggca

SEQ ID NO: 52 - DNA, 26 bp, Artificial Sequence
  rrs-W514 Oligonucleotide
gcaccctacgtattaccgcggctgct

SEQ ID NO: 53 - DNA, 26 bp, Artificial Sequence
  rrs-Q514 Oligonucleotide
gcactctacgtattaccgcggctgct

SEQ ID NO: 54 - DNA, 26 bp, Artificial Sequence
  rrs-W1401 Oligonucleotide
tgacgtgacgggcggtgtgtacaagg

SEQ ID NO: 55 - DNA, 26 bp, Artificial Sequence
  rrs-Q1401 Oligonucleotide
tgacgtgacgggcggtatgtacaagg

SEQ ID NO: 56 - DNA, 30 bp, Artificial Sequence
  rrs-W1484 Oligonucleotide
gacttcgtcccaatcgccgatcccaccttc

SEQ ID NO: 57 - DNA, 30 bp, Artificial Sequence
  rrs-Q1484 Oligonucleotide
gacttcgtcccaatcgccgatcctaccttc

SEQ ID NO: 58 - DNA, 33 bp, Artificial Sequence
  rrl control Oligonucleotide
gtgttaccactgactggtacggctaccttcctg